(19) United States
(12) Patent Application Publication
(10) Pub. No.: US 2019/0117426 A1
(43) Pub. Date: Apr. 25, 2019

(54) JOINT FOR AN ORTHOPEDIC DEVICE

(71) Applicant: OTTOBOCK SE & CO. KGAA, Duderstadt (DE)

(72) Inventor: Marcus LURSSEN, Gottingen (DE)

(73) Assignee: OTTOBOCK SE & CO. KGAA, Duderstadt (DE)

(21) Appl. No.: 16/098,374

(22) PCT Filed: Apr. 26, 2017
(86) PCT No.: PCT/EP2017/059917

§ 371 Date: Nov. 1, 2018

(30) Foreign Application Priority Data
May 2, 2016 (DE) 102016108107.8

(51) Int. Cl. A61F 5/01

(52) U.S. Cl. CPC A61F 5/0102; A61F 2005/0132

(57) ABSTRACT

A joint for an orthopedic device, which comprises a first joint arm with a recess, a second joint arm that projects into said recess and is connected therein to the first joint arm so as to pivot about a pivot axis, and at least one spacing element that is arranged inside the recess between the first joint arm and the second joint arm and through which the pivot axis runs, wherein the at least one spacing element is accessible from outside, and particularly projects out of the recess.

The invention relates to a joint for an orthopedic device, comprising a first articulated arm having a recess, a second articulated arm, which projects into the recess and is connected therein to the first articulated arm so as to be pivotable about a pivot axis, and at least one spacer element, which is arranged inside the recess between the first articulated arm and the second articulated arm and through which the pivot axis extends.

Joints of this kind are used in a large number of orthopedic devices, for example ankle braces. The first articulated arm has a recess into which the second articulated arm protrudes. As a result, the side delimiting walls of the recess can be used as stop elements by which a pivot movement of the two articulated arms relative to one another can be limited. It goes without saying, however, that it is also possible to use additional stop elements and/or damping elements. Between the first articulated arm and second articulated arm, at least one spacer element, for example a washer, is generally arranged within said recess.

Often, the two articulated arms each have a hole, which must be overlapped in order to assemble the joint. In this position, a connecting element, for example a bolt, a pin or a screw, can be guided through the holes in the two articulated arms. The pivotable connection of the two articulated arms to one another is thus established. To compensate for production tolerances and/or prevent the two articulated arms from abutting one another across the entire surface area, at least one spacer element, for example in the form of a washer, is arranged within the recess. To assemble the joint, this washer has to be positioned within the recess such that the connecting element can be guided not only through the holes in the two articulated arms, but also through a hole provided for this purpose in the washer. To do so, the washer must therefore be moved relative to the first articulated arm and relative to the second articulated arm. However, this must occur within the recess, yet it can only be done with difficulty and entails high complexity due to very confined spaces and the poor accessibility.

The problem addressed by the invention is therefore to develop a generic joint according to the preamble of claim 1 such as to make assembly simpler and quicker.

The invention solves the stated problem by a joint according to the preamble of claim 1, characterized in that the at least one spacer element is accessible from the outside, in particular protrudes from the recess. In this way, a part of the spacer element is not only visible, but can also be gripped manually or using a tool and moved and secured in a desired position. This makes it much simpler to position the spacer element and can speed up the production process for the joint.

The recess may comprise an edge having, for example, a convex shape; for example, it may bulge in the direction towards the second articulated arm. However, this is not strictly necessary. Thus it is also conceivable, for example, that one or more indentations or pockets forming a concave part of the edge are formed in said contour of the edge. In this case, it is possible to design the spacer element such that it is accessible from the outside but does not protrude beyond a convex rounding-off of the contour of the edge of the recess. Of course, this design is still part of the invention since the spacer element is accessible from the outside.

Preferably, the joint has two spacer elements arranged in the recess on different sides of the second articulated arm. In this case, the two spacer elements are preferably designed to protrude out of the recess in the first articulated arm so that the positioning and securing during assembly can be ensured in such a way that any connecting element to be inserted can be inserted easily, readily and securely.

In a preferred embodiment, at least one of the spacer elements has a stop that prevents the spacer element from being able to be fully received between the first articulated arm and second articulated arm in the recess. A stop of this kind can be, for example, an end piece arranged at an angle or a thickened portion. Preferably, all the spacer elements used have a stop of this kind.

Preferably, the spacer element is mounted non-rotatably relative to the first articulated arm. This can be achieved particularly simply by the stop that abuts the first articulated arm, for example at the edge of the recess, and thus prevents the spacer element from rotating relative to said articulated arm. Alternatively, of course, it is also possible, though technically more complex to implement, to non-rotatably mount the at least one spacer element on the second articulated arm.

This prevents one of the spacer elements from inadvertently being fully received between the two articulated arms in the recess during assembly and thus from being unable to be subsequently positioned or secured.

Advantageously, the stop is formed such as to follow a contour of the edge and in particular such as to abut the contour in a positive manner.

Preferably, the joint has a connecting element, by which the first articulated arm is connected to the second articulated arm. Advantageously, the at least one spacer element also has a hole, through which the connecting element extends when the joint is assembled. By means of a connecting link of this kind, which can be formed for example as a screw, bush, bolt or pin, both the pivotable connection between the two articulated arms and the relative positioning of the components used are ensured simultaneously.

It has proven advantageous for the stop to be formed on at least one of the spacer elements such as to abut an edge of the recess when the joint is assembled. This design has many advantages. Firstly, it ensures that the spacer element is specifically as large as necessary so as to not be fully received in the recess. This means that the spacer element can be as small as possible and thus formed using as little material as possible. At the same time, by means of the stop that abuts the edge of the recess, the at least one spacer element can be positioned during assembly in a particularly simple manner. The at least one spacer element merely must be pushed into the recess between the two articulated arms until a stop abuts the edge of the recess. This allows at least for pre-positioning, such that a subsequent fine adjustment of the position need only involve a small displacement or can be omitted entirely.

It has proven advantageous if the at least one spacer element consists of a plastics material. This can prevent rattling noises within the joint, which may otherwise occur in the case of metal articulated arms, for example. At the same time, frictional resistance when the joint pivots is reduced, and the joint is perceived as having smooth operation, in particular when used in an orthopedic device.

The invention also solves the stated problem by means of an orthopedic device comprising at least one joint described herein. Advantageously, the orthopedic device has two such joints, which preferably can be arranged on different sides of a body part, in particular of a joint. Preferably, the orthopedic device is a rail system for an orthosis or an ankle brace. It goes without saying, however, that the joint can also be used in other orthopedic devices.

The spacer element for a joint according to the invention should have optimum sliding properties, i.e. as low a coefficient of friction as possible, and should also be as sturdy as possible, i.e. resistant to wear. For this purpose, the spacer can be coated, for example, or can be made of a material with very good sliding properties. In particular when a coating is used, it is possible to simultaneously optimize the sliding properties and, for example, the resistance to wear and the abrasion resistance.

The joints for orthopedic devices according to the present invention can be used for orthoses and prostheses for many different body parts. They can be used as knee, ankle, hip, elbow or wrist joints, or also at various positions of orthoses and/or prostheses.

An embodiment of the present invention will be described in more detail below by means of the accompanying drawings, in which:

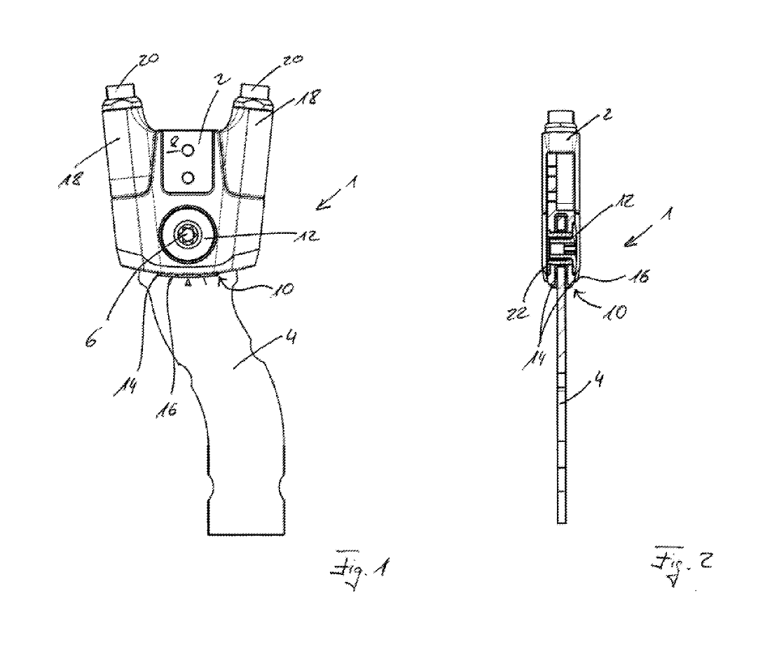

FIG. 1 is a schematic view of a joint according to a first embodiment of the present invention,

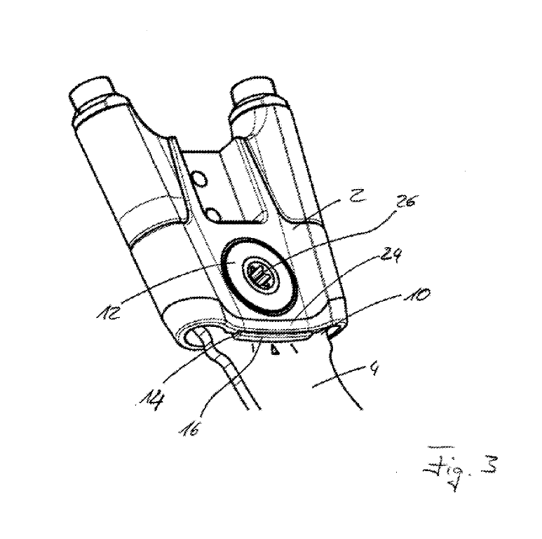

FIG. 2 is a sectional view of the joint from FIG. 1,

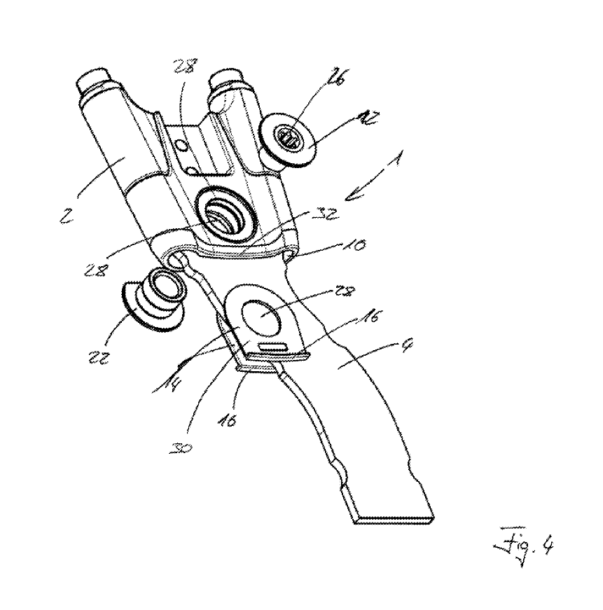

FIG. 3 is a schematic three-dimensional view of an enlarged portion of the joint from FIGS. 1 and 2,

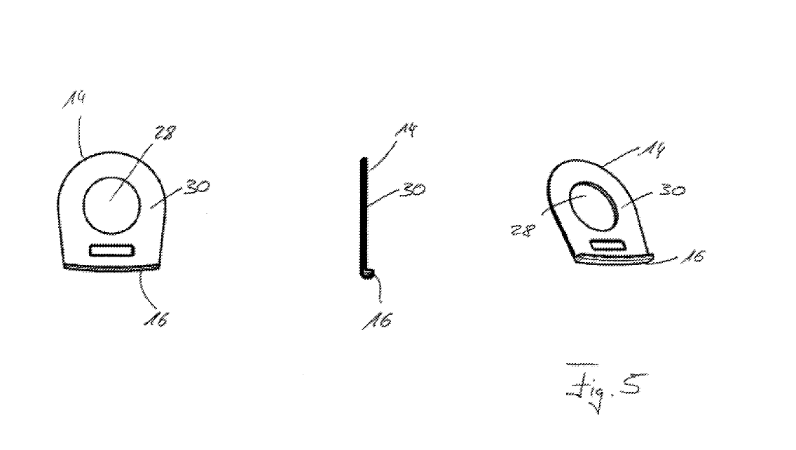

FIG. 4 shows the joint from FIGS. 1 to 3 in a partly disassembled state, and

FIG. 5 shows a spacer element according to an embodiment of the present invention in three different views.

FIG. 1 shows a joint 1 according to a first embodiment of the present invention. Said joint has a first articulated arm 2 and a second articulated arm 4, which are interconnected so as to be pivotable about a pivot axis 6. The first articulated arm 2 has a receiving device 8, on which a further component, for example a rail for an orthopedic device, can be arranged.

The first articulated arm 2 also has a recess 10 into which the second articulated arm 4 protrudes. By means of the connecting element 12 guided through the two articulated arms 2, 4, the articulated arms 2 and 4 are interconnected.

In FIG. 1, within the recess 10, a spacer element 14 can be seen between the first articulated arm 2, i.e. the upper wall of the recess 10, and the second articulated arm 4. Said element protrudes out of the recess 10 by means of a stop 16.

In addition, two receiving bushes 18 are arranged on the first articulated arm 2, into which bushes it is possible to insert a for example resilient stop element which defines the pivot angle about which the second articulated arm 4 is pivotable relative to the first articulated arm 2. By means of adjustment devices 20, the toughness of a stop of this kind can be adjusted.

FIG. 2 is a sectional view of the joint 1. The first articulated arm 2 and the second articulated arm 4 can be seen, as well as the connecting element 12 guided through the two articulated arms 2, 4. From the left-hand side of the first articulated arm 2 in FIG. 2, a counterpart-connecting element 22 is inserted into the holes provided for that purpose and is screwed to the connecting element 12. This ensures the two articulated arms 2, 4 are fastened to one another. On both sides of the second articulated arm 4 within the recess 10, there is a spacer element 14, said elements abutting an edge of the recess 10 by means of the respective stops 16.

FIG. 3 is an enlarged illustration in a schematic three-dimensional view. In this figure, the recess 10 into which the second articulated arm 4 is pushed can be clearly seen. It is also possible to see the stop 16 on the connecting element 12, which is also pushed into the recess 10. Said element is located between the first articulated arm 2 and the second articulated arm 4. This means that the second articulated arm 4 is located on one side of the connecting element 12 and at least a part of the first articulated arm 2, in this case specifically an upper wall 24 of the recess 10, is located on the other side. Similarly, on the opposite side of the second articulated arm 4, a lower wall of the recess 10, also belonging to the first articulated arm 2, can of course be located on the side of the spacer element 14 opposite the second articulated arm 4.

FIG. 3 shows that the connecting element 12 is equipped with a positive-fit element 26, by means of which said element can be operated and handled using a tool.

FIG. 4 shows the joint 1 in a partly disassembled state. The first articulated arm 2 has a recess 10 into which the second articulated arm 4 is pushed. However, the two spacer elements 14 are not inserted into the recess 10. It can be seen that they have a hole 28 through which the connecting element 12 and, in the embodiment shown, also the counterpart-connecting element 22 can be guided. The counterpart-connecting element 22 has an internal thread designed to correspond to an external thread of the connecting element 12. The positive-fit element 26 is shown again in the connecting element 12.

The two spacer elements 14 again have the stops 16, which, in the embodiment shown, form an angle with the main surface 30 of each spacer element 14, said angle being 90° in the present case. The stop 16 is formed such that it abuts an edge 32 of the recess 10 when, as shown in FIGS. 1, 2 and 3, the spacer element 14 is pushed into the recess 10 and the connecting element 12 and the counterpart-connecting element 22 are guided through the hole 28. It goes without saying that, for example, a nut for a screw or a similar element is possible instead of a counterpart-connecting element.

It can be seen in FIG. 4 that the first articulated arm 2 and the second articulated arm 4 each comprise a hole 28, which have to be overlapped in order to assemble the joint 1. Only then can the connecting element 12 be guided through the holes 28 in the first articulated arm 2, the second articulated arm 4 and all the spacer elements 14 provided.

FIG. 5 shows the spacer element 14 in three different views. It can be seen in each view that the spacer element 14 comprises a main surface 30 in which the hole 28 is arranged. The spacer element 14 further has the stop 16, which bulges out slightly in order to follow the edge 32 of the recess 10. Of course, other geometries are also possible in this case.

### LIST OF REFERENCE NUMERALS

1 Joint
2 First articulated arm
4 Second articulated arm
6 Pivot axis
8 Receiving device
10 Recess
12 Connecting element
14 Spacer element
16 Stop
18 Receiving bush
20 Adjustment device
22 Counterpart-connecting element
24 Upper wall
26 Positive-fit element
28 Hole
30 Main surface
32 Edge

1. A joint for an orthopedic device, comprising:
a first articulated arm having a recess;
a second articulated arm, which protrudes into the recess and is connected therein to the first articulated arm so as to be pivotable about a pivot axis; and
at least one spacer element, which is arranged between the first articulated arm and the second articulated arm within the recess and through which the pivot axis extends;
wherein the at least one spacer element is accessible from outside of the recess and protrudes out of the recess.

2. The joint according to claim 1, wherein the joint has at least two spacer elements arranged in the recess on different sides of the second articulated arm.

3. The joint according to claim 1, wherein at least one of the spacer elements has a stop that prevents the spacer element from being able to be fully received between the first articulated arm and the second articulated arm in the recess.

4. The joint according to claim 1, wherein the at least one spacer element is non-rotatably mounted relative to the first articulated arm.

5. The joint according to claim 1, wherein the at least one spacer elements has a hole through which a connecting element extends, the connecting element connecting the first articulated arm and the second articulated arm.

6. The joint according to claim 3, wherein the stop is formed such as to abut an edge of the recess.

7. The joint according to claim 6, wherein the stop is formed such as to follow a contour of the edge and to abut the contour in a positive manner.

8. The joint according to claim 1, wherein the at least one spacer element comprises a plastics material.

9. An orthopedic device comprising at least one joint according to claim 1.

10. The orthopedic device according to claim 9, wherein the device is a rail system for an orthosis or an ankle brace.

11. A joint for an orthopedic device, comprising:
a first articulated arm having a recess;
a second articulated arm, which protrudes into the recess and is pivotally connected to the first articulated arm about a pivot axis; and
at least one spacer element, which is arranged between the first articulated arm and the second articulated arm within the recess and through which the pivot axis extends, the at least one spacer element protruding out of the recess.

12. The joint according to claim 11, wherein the at least one spacer element includes at least two spacer elements arranged in the recess on opposite sides of the second articulated arm.

13. The joint according to claim 11, wherein the at least one spacer element comprises a stop that prevents the at least one spacer element from being fully insertable between the first articulated arm and the second articulated arm within the recess.

14. The joint according to claim 11, wherein the at least one spacer element is non-rotatably mounted relative to the first articulated arm.

15. The joint according to claim 11, wherein the at least one spacer element has a hole through which a connecting element extends, the connecting element connecting the first articulated arm and the second articulated arm.

16. The joint according to claim 13, wherein the stop is arranged to abut an edge of the recess.

17. The joint according to claim 16, wherein the stop is formed to follow a contour of the edge of the recess and to abut the contour.

18. The joint according to claim 11, wherein the at least one spacer element comprises a plastic material.
